(19) United States
(12) Patent Application Publication
(10) Pub. No.: US 2023/0044320 A1
(43) Pub. Date: Feb. 9, 2023

(54) BIOPRINTING SYSTEM

(71) Applicant: Inventia Life Science Pty Ltd, Alexandria, New South Wales (AU)

(72) Inventors: Gregoire Andrew Francis CHALONY, Rueil Malmaison (FR); Alberto PILONI, Newtown, New South Wales (AU); Zachary Benjamin ARTIST, Maroubra, Victoria (AU); Samuel James MYERS, Blackheath, New South Wales (AU); Andrew SEXTON, Alexandria, New South Wales (AU); Aidan Patrick O'MAHONY, Randwick, New South Wales (AU); William Wen-Feng LIM, Gordon, New South Wales (AU); Deanna Maree HOOD,, Helensvale, Queensland (AU)

(73) Assignee: Inventia Life Science Pty Ltd, Alexandria, New South Wales (AU)

(21) Appl. No.: 17/782,915

(22) PCT Filed: Dec. 7, 2020
(86) PCT No.: PCT/AU2020/051333

§ 371 Date: Jun. 6, 2022

(30) Foreign Application Priority Data
Dec. 6, 2019 (AU) 2019904627

(51) Int. Cl. A61L 26/00; A61M 35/00; B25J 9/16; B25J 13/08; G01S 13/08

(52) U.S. Cl. CPC A61L 26/008; A61L 26/0057; A61M 35/00; B25J 9/1694; B25J 13/086; G01S 13/08

(57) ABSTRACT

The present disclosure provides a bioprinting system (100) for printing a liquid directly onto a subject. The bioprinting system (100) comprises a bioprinting assembly (102). Optionally, a robotic arm (104) and a control system (150) are provided. The bioprinting assembly (102) may be coupled to the robotic arm (104) to be positionable relative to the subject. The bioprinting assembly (102) is configured to dispense the liquid onto the subject and comprises a reservoir (120) for holding the liquid and a loading mechanism (134) to prime the reservoir (120) with the liquid directly prior to printing. The loading mechanism (134) has a one way inlet to permit liquid to be loaded into the reservoir (120) and prevent fluid from exiting the reservoir via the one way inlet. There is also provided associated methods.

### TECHNICAL FIELD

The technology relates to a bioprinting system that is capable of printing cells on a site of a subject to treat or dress a wound.

### BACKGROUND

Applying hydrogels that include cells and/or medicaments over a wound of a patient is known. Such known methods of applying hydrogels do not apply the hydrogel evenly over the wound, which may result in an inconsistent deposition of the cells and/or medicaments over the wound. This may result in less than optimal healing of the wound and more hydrogel being applied over the wound than is necessary.

The present inventors have developed a bioprinting system suitable for printing cells and materials over a wound of a subject to form a hydrogel.

### SUMMARY

According a first aspect, there is provided a bioprinting system for printing a liquid to a site of a subject, the bioprinting system comprising:

a bioprinting assembly configured to dispense the liquid to the site of the subject, the bioprinting assembly having at least one reservoir configured to hold the liquid to be dispensed by the bioprinting assembly, and a loading mechanism in fluid communication with the reservoir configured to load the reservoir with the liquid prior to printing onto the site of the subject, wherein the loading mechanism comprises a one way inlet that permits the liquid to be loaded into the reservoir and prevents fluid from exiting the reservoir via the one way inlet.

In an embodiment, the subject is a patient, the site is a wound in the subject's skin, and the liquid dispensed by the bioprinting assembly forms a gel over the wound.

The term ‘liquid’ as used herein may refer to any substance which is to be printed onto the site on a subject using the described bioprinting assembly. For example, the liquid may include any one or more of bio-ink, cell-ink, activator, medicament, or other substance.

The features and embodiments described in the present disclosure may provide a number of advantages. For example, the loading mechanism may enable bio-inks and activator solutions containing cells to be loaded directly into the reservoir in a manner that facilitates an optimised workflow for the respective clinician(s) in a clinical environment. Further possible advantages are as follows:
The clinician may directly load liquid containing cells into the reservoir of the bioprinting system at a time directly before printing onto a wound. This may be particularly advantageous when printing autologous cells, as maintaining high cell viability is critical to the outcome of the treatment. For example, the source of autologous cells may be a skin graft that is taken from the patient during the same procedure as a bio-ink treatment is applied.
Loading of bio-inks or activators containing cells into the reservoir at a time directly before printing onto a wound allows greater flexibility for the clinician to decide intra-operatively the type and volume of bio-inks or activators that should be applied to the wound upon examination of the wound.
Loading cells directly into the reservoir via the loading mechanism may enable the component surface area in contact with the patient's cells to be minimised, which may reduce losses due to dead volume.
Loading the liquids into a reservoir contained within the bioprinting assembly may ensure that all of the fluidic connections in the bioprinting assembly can be tested prior to the procedure. This may further streamline the workflow and has the potential to increase the reliability of the system.
Loading directly into a reservoir of the bioprinting assembly removes the need for complex loading architecture within the bioprinting system, which can reduce possibilities of failure modes associated with such complex architecture and may improve the reliability of the system.
Preparing cells and biomaterials for a treatment may be performed in a sterile operating theatre and loading directly into the reservoir which assists in preventing a contamination (of either pathogens or particulates) from being introduced into the system. This may remove the need for a pre-prepared cartridge of the biomaterials or cells.

According to embodiments, the loading mechanism provides a sterile fluidic connection. The loading mechanism may comprise any one or more of a check valve, a septum, and a luer lock

According to embodiments, the loading mechanism has a priming fluid line providing a fluid communication between the one way inlet and the reservoir.

According to embodiments, the one way inlet is configured to be removably coupled to a loading device, such as a syringe. Preferably, the one way inlet has a connector configured to be removably coupled to the loading device. The connector may comprise any one or more of a septum, a check valve, and a luer lock. The coupling of the one way inlet to the loading device is preferably a sterile fluidic connection.

According to embodiments, the bioprinting assembly comprises a plurality of reservoirs. The bioprinting assembly may comprise a plurality of loading mechanisms each in fluid communication with a respective reservoir.

According to embodiments, the bioprinting system further comprises a robotic arm coupled to the bioprinting assembly, the robotic arm configured to move and position the bioprinting assembly over the site.

According to embodiments, the bioprinting system further comprises a control system configured to control the bioprinting assembly and/or the robotic arm.

According to embodiments, the bioprinting assembly further comprises a distance sensor configured to monitor the distance between the bioprinting assembly and the site of the subject. The control system is preferably configured to use distance information from the distance sensor to control the robotic arm to maintain the bioprinting assembly at a predetermined distance from the site while printing the liquid.

In an embodiment, the bioprinting assembly further comprises an aiming aid that is configured to assist with positioning the bioprinting assembly.

In an embodiment, the aiming aid is a laser.

In an embodiment, the bioprinting system further comprises a controller configured to move and position the bioprinting assembly by controlling the robotic arm.

In an embodiment, the bioprinting assembly further comprises at least one reservoir configured to hold a liquid to be dispensed by the bioprinting assembly.

In an embodiment, the at least one reservoir has a priming fluid line configured to enable loading and/or priming of the at least one reservoir.

In an embodiment, the priming fluid line has a connector that is configured to be removably coupled to a syringe. According to embodiments, the connector is or comprises a septum, a check valve, or a luer lock.

In an embodiment, the at least one reservoir has a dispensing fluid line that is configured to dispense fluid from the at least one reservoir.

In an embodiment, the dispensing fluid line has a dispensing outlet having:

an open configuration that allows liquid to be dispensed from the at least one reservoir; and

a closed configuration that prevents liquid from being dispensed from the at least one reservoir.

In an embodiment, the dispensing outlet is a nozzle or valve.

In an embodiment, the bioprinting system further comprises a pressure regulating system coupled in fluid communication with the at least one reservoir, the pressure regulating system configured to regulate pressure within the at least one reservoir.

In an embodiment, the pressure regulating system is configured to be coupled to a source of pressurized gas.

In an embodiment, the source of pressurized gas is an air compressor.

According to embodiments, the robotic arm is a six-axis or seven-axis robotic arm. In an embodiment, the robotic arm is a six-axis robotic arm. In an embodiment, the robotic arm is a seven-axis robotic arm.

In an embodiment, the robotic arm is configured to be manually moved by a user to move and position the bioprinting assembly.

In an embodiment, the liquid to be dispensed from the bioprinting assembly includes reagents and activators.

In an embodiment, the liquid to be dispensed from the bioprinting assembly is selected from bio-inks, radiation curable bio-inks, activators, cell-inks, and cell-culture solutions.

In an embodiment, the bioprinting assembly further comprises a radiation source configured to cure a radiation curable fluid dispensed by the bioprinting assembly.

In an embodiment, the radiation source is a UV radiation source.

In an embodiment, the UV radiation source is an array of UV LEDs.

According to embodiments, the robotic arm and the bioprinting assembly are configured such that the bioprinting assembly can be manoeuvred to print the liquid onto the site of the subject in any desired orientation. For example, the bioprinting assembly may print in an upwards orientation towards an underside of a subject, in a sideways orientation onto the side of a subject, downwards onto the upper side of a subject or any orientation between these.

According to a second aspect, there is provided a method of forming a gel over a wound of a subject using the bioprinting system of the first aspect, the method comprising:

a) dispensing a reagent from the bioprinting assembly to a point of the site;

b) dispensing an activator from the bioprinting assembly onto the dispensed reagent to form a hydrogel; and

c) repeating steps a) and b) at a plurality of different points of the site to form the gel over the wound.

In an embodiment, the reagent is selected from bio-inks, radiation curable bio-inks, activators, cell-inks, and cell-culture solutions.

According to a third aspect, there is provided a method of forming a gel over a wound of a subject using the bioprinting system of the first aspect, the method comprising:

a) dispensing a radiation curable reagent from the bioprinting assembly to a point of the site;

b) illuminating the dispensed radiation sensitive reagent with the radiation source to form a hydrogel; and

c) repeating steps a) and b) at a plurality of different points of the site to form the gel over the wound.

In an embodiment, the radiation curable reagent is a radiation curable bio-ink.

In an embodiment, the radiation curable bio-ink is a UV curable bio-ink.

According to a fourth aspect, there is provided a method of printing liquid to a site of a subject using the bioprinting assembly of the first aspect, the method comprising:

a) dispensing liquid from the bioprinting assembly to a point of the site; and

b) repeating step a) at a plurality of different points of the site to cover the site with the liquid.

In an embodiment, the liquid includes cells and/or medicaments.

According to embodiments, the bioprinting assembly is manoeuvred in any desired orientation to dispense the liquid onto the point of the site in a predetermined orientation. According to embodiments, a droplet size and/or droplet volumes of the liquid is selected such that the liquid forms a gel at the site of the subject without movement due to gravity. For example, the droplet volume may be from 0.5 to 500 nanolitres, 0.5 to 200 nanolitres, 0.5 to 100 nanolitres, 0.5 to 50 nanolitres, 0.5 to 10 nanolitres, 0.5 to 5 nanolitres, 5 to 10 nanolitres, 10 to 50 nanolitres, 10 to 100 nanolitres, 5 to 500 nanolitres, 10 to 500 nanolitres, 50 to 500 nanolitres, 100 to 500 nanolitres, 250 to 500 nanolitres or any other suitable size/volume.

According to a fifth aspect, there is provided a use of the bioprinting system of the first aspect to print liquid to a site of a subject.

According to a sixth aspect, there is provided a method of loading a reservoir with a liquid, comprising: a) providing the bioprinting system of the first aspect; b) connecting a container and/or loading device comprising the liquid to the loading mechanism in a sterile fluidic connection; c) transferring the liquid from the container to the bioprinting assembly; and d) loading the reservoir with the liquid.

According to embodiments, the liquid is a bio-ink or cell-ink and comprises cells. The cells may be autologous cells of the subject.

According to embodiments, the container and/or loading device is a syringe, and wherein transferring the liquid comprises injecting the liquid into the bioprinting assembly.

According to embodiments, the bioprinting system is provided in an operating theatre, and wherein the steps b) to d) are each performed within the operating theatre before the liquid is to be printed onto the site of the subject

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this specification.

### BRIEF DESCRIPTION OF THE DRAWINGS

Preferred embodiments of the present invention will now be described, by way of examples only, with reference to the accompanying drawings, in which:

FIG. 1 is a front isometric view of a bioprinting system according to an embodiment of the present invention;

FIG. 2 is a front view of the bioprinting assembly and the robotic arm of the bioprinting system of FIG. 1;

FIG. 3 is a front view of the bioprinting assembly of FIG. 2 with the access panel removed;

FIG. 4 is a bottom view of the bioprinting assembly of FIG. 2;

FIG. 5 is a rear isometric view of the bioprinting system of FIG. 1;

FIG. 6 is a block diagram illustrating the control system of the bioprinting system of FIG. 1;

FIG. 7 shows a wound of a patient over which the bioprinting system of FIG. 1 may form a gel;

FIG. 8 shows a gel that is to be formed by the bioprinting system of FIG. 1;

FIG. 9 shows another wound of a patient over which the bioprinting system of FIG. 1 may form a gel; and

FIG. 10 is a three-dimensional plot of a scan using a distance sensor.

### DETAILED DESCRIPTION OF EMBODIMENTS

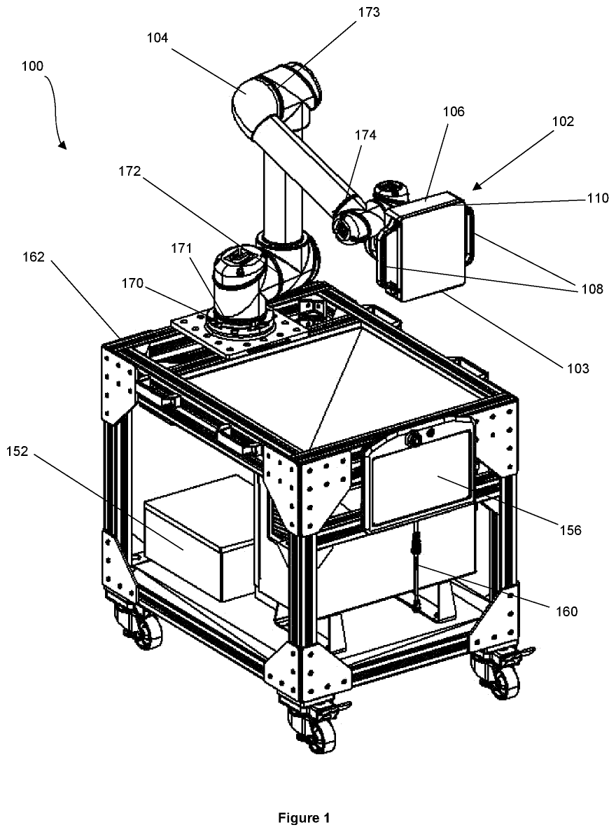

FIG. 1 shows a bioprinting system 100 according to an embodiment of the present invention. The bioprinting system 100 has a bioprinting assembly 102 removably coupled to a robotic arm 104. The robotic arm 104 is a six-axis robotic arm, however, any other suitable robotic arms known in the art may also be used. For example, the robotic arm 104 may be replaced with a seven-axis robotic arm.

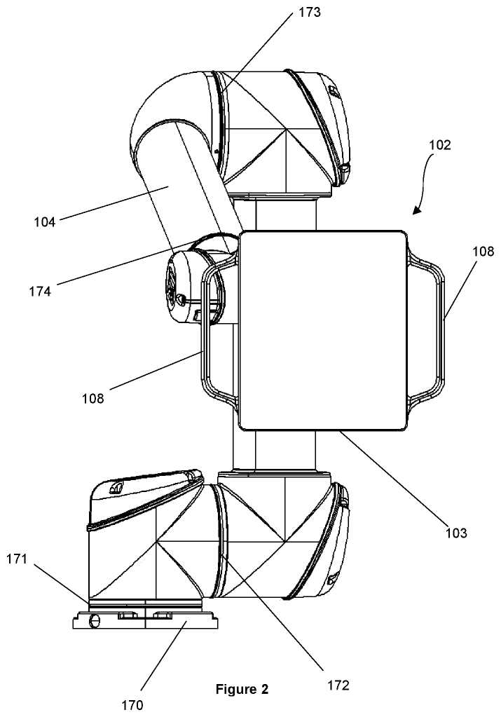

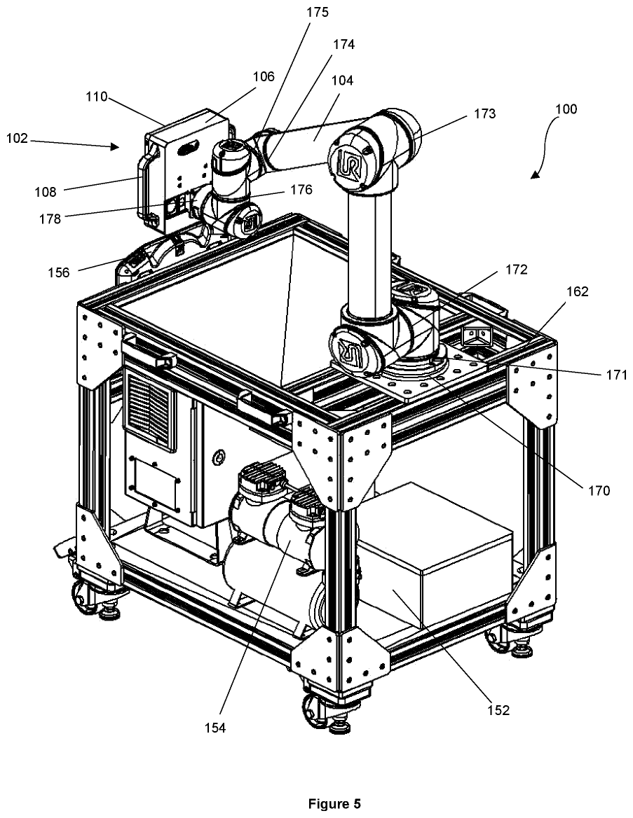

The various components of the bioprinting system may be housed in any desired manner. For example, they may be attached to or located on/within a static structure or may be attached to or located on/within a mobile structure, such as the trolley 162 shown in FIGS. 1 and 5. The trolley 162 allows movement of the bioprinting system 100 to a desired location. The robotic arm 100 is attached to the trolley 162 via the mounting base 170 of the robotic arm. According to other embodiments, the robotic arm could be mounted on another surface or at a fixed location. The robotic arm 100 is moveable about the six axes of rotation defined by locations 171-176 such that the bioprinting assembly 102 may manoeuvred and oriented as desired. FIG. 2 shows the robotic arm 104 and bioprinting assembly 102 when not attached to the trolley 162. As shown in FIG. 5 the bioprinting assembly 102 is mounted to the robotic arm 104 via the mounting connector 178 of the robotic arm 104.

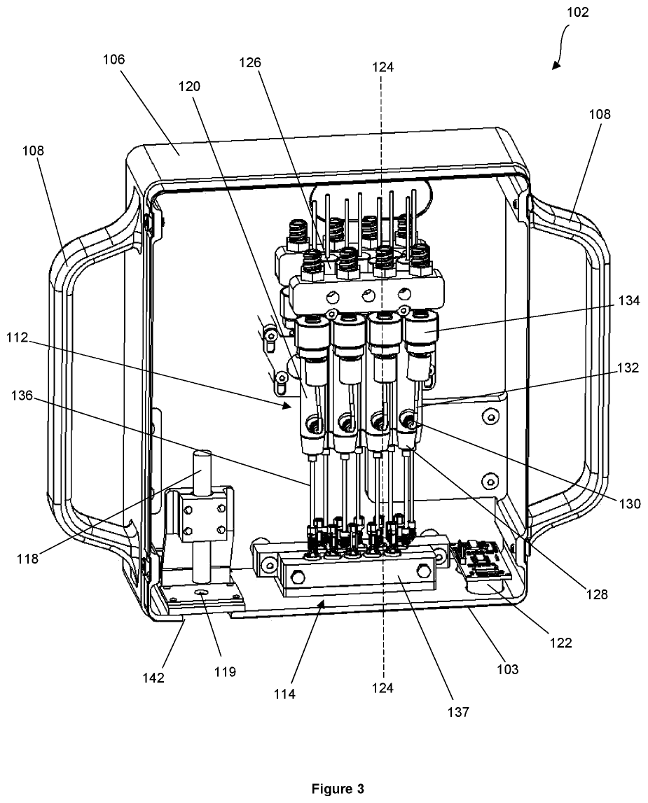

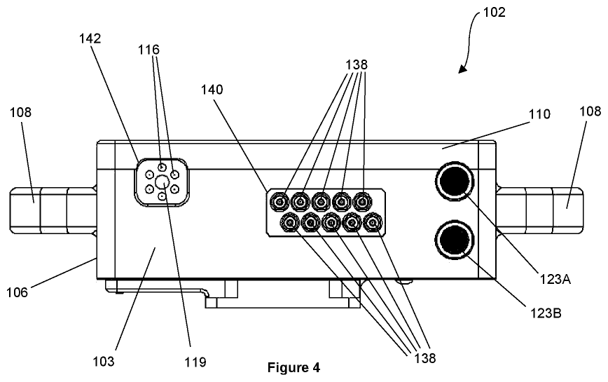

Referring to FIGS. 2 to 4, the bioprinting assembly 102 has a printhead housing 106 having handles 108 and an access panel 110. Removing the access panel 110 permits access to the inside of the printhead housing 106. Disposed within the printhead housing 106 is a set of reservoirs 112, a dispensing system 114, a radiation source 116, and an aiming aid 118. The set of reservoirs 112 has eight reservoirs 120, with a row of four reservoirs 120 visible in FIG. 3 and a row of four reservoirs 120 hidden behind the visible reservoirs 120. However, the set of reservoirs 112 may have any desired number of reservoirs 120. As shown in FIG. 4, there are ten available dispensing outlets 138, which means the embodiment shown may include up to ten reservoirs 120. Each reservoir 120 may contain a respective fluid or liquid, alternatively the more than one reservoir 120 may contain the same fluid or liquid.

The radiation source 116 is in the form of an array of ultraviolet (UV) light emitting diodes (LEDs) as shown in FIG. 4. The radiation source 116 is configured to cure a UV curable liquid such as, for example, a photosensitive bio-ink or a UV curable bio-ink. It is also envisaged that the radiation source 116 may be any other suitable radiation source known in the art that is capable of curing a radiation-curable liquid. For example, the radiation-curable liquid may comprise hyaluronic acid, gelatin, polyethylene glycol (PEG), and/or collagen, each of which may be modified with acrylate, methacrylate, and/or norbornene. Specific examples of a radiation-curable liquid may include methacrylated hyaluronic acid, methacrylated gelatin, PEG diacrylate, PEG dimethacrylate, methacrylated collagen.

The aiming aid 118 is in the form of a laser that is used as a visual aid to position the bioprinting assembly 102. Aperture 119 is provided to enable the laser beam of the aiming aid 118 to exit the printhead housing 106. It is also envisaged that the aiming aid 118 may be any other suitable means known in the art that can be used as a visual aid to position the bioprinting assembly 102.

Within the printhead housing 106 is a distance sensor 122 that is configured to monitor the distance between the base 103 of the bioprinting assembly 102 and a printing surface. The printing surface may be the surface of a subject, for example, the surface of a patient's skin. It is also envisaged that the distance sensor 122 may be disposed outside and coupled to the printhead housing 106. The distance sensor 122 may be an ultrasonic sensor, an optical sensor, a camera, an inductive sensor, a capacitive sensor, a photoelectric sensor, a contact sensor that physically contacts the surface of a patient's skin, or any other suitable sensor known in the art that is capable of monitoring the distance between the base 103 of the bioprinting assembly 102 and the printing surface. The distance sensor 122 may have an emitting portion 123A configured to emit a signal/wave and a receiving portion 123B configured to receive the emitted signal/wave, which are exposed through the base 103 of the bioprinting assembly 102 in the embodiment shown in FIG. 4.

Referring to FIG. 3, each reservoir 120 has a longitudinal axis 124 extending substantially vertically, a cap 126 located at the top of the reservoir 120, a reservoir outlet 128 located at a lower region of the reservoir 120, and a reservoir inlet 130 located at a predetermined height above the reservoir outlet 128. For each reservoir 120, the cap 126, the reservoir outlet 128, and the reservoir inlet 130 are all in fluid communication with the interior of the reservoir 120.

Coupled in fluid communication to the reservoir inlet 130 of each reservoir 120 is a priming fluid line 132. Each priming fluid line 132 has a connector 134 that allows a syringe, or the like, to be removably coupled to the priming fluid line 132. Each connector 134 may provide any suitable type of connection means, for example the connector 134 may be or comprise a luer lock. Coupling a syringe, or the like, to the connector 134 of any of the priming fluid lines 132 allows the content of the syringe, or the like, to be injected into the respective reservoir 120. Although the priming fluid lines 132 have been described and illustrated as having connectors 134, it is also envisaged that any other suitable means known in the art that permits a syringe, or the like, to be removably coupled to the priming fluid lines 132 may be used. One or more of the connector 134, priming fluid line 132 and reservoir inlet 130 may form part of a loading mechanism. The loading mechanism preferably providing a sterile fluidic connection between the reservoir 120 and a container, such as a syringe or the like, which provides the liquid to the reservoir 120. The loading mechanism preferably has a one way inlet which permits the liquid to be loaded into the reservoir 120 but when prevents any fluid from escaping. The connector 134 may be or include the one way inlet. The one way inlet may therefore maintain a pressure within the bioprinting assembly without letting any liquid or gas escape.

The dispensing system 114 comprises a plurality of dispensing fluid lines 136, each of which are coupled in fluid communication to the reservoir outlet 128 of one of the reservoirs 120. Coupled in fluid communication to each dispensing fluid line 136 is a dispensing outlet 138 in the form of a nozzle having a normally closed configuration and an open configuration. The dispensing outlets 138 are housed in housing 137 within the printhead housing 106 adjacent to the base 103. For each dispensing fluid line 136, when the dispensing outlet 138 is in the open configuration, fluid is allowed to flow out of the respective reservoir 120 through the reservoir outlet 128 and through the dispensing fluid line 136 to be dispensed from the dispensing outlet 138. For each dispensing fluid line 136, when the dispensing outlet 138 is in the closed configuration, fluid is prevented from being dispensed from the dispensing outlet 138. It is envisaged that each dispensing outlet 138 may be a micro-solenoid valve, however, any other suitable valves/nozzles known in the art may also be used.

For each reservoir 120, the volume of the dispensing fluid line 136 and the volume between the reservoir outlet 128 and the reservoir inlet 130 within the reservoir 120 define a predetermined volume. The predetermined volume can be increased or decreased by increasing or decreasing the height difference between the reservoir outlet 128 and the reservoir inlet 130 for each reservoir 120, respectively. The predetermined volume can also be increased or decreased by increasing or decreasing the volume of the dispensing fluid line 136. It will be appreciated that increasing the predetermined volume will reduce, or possibly prevent, liquid flowing from within the reservoir 120 back out the respective priming fluid line 120.

Referring to FIG. 4, the dispensing outlets 138 are aligned with a hole 140 in the printhead housing 106 such that each dispensing outlet 138 is configured to dispense fluid out of the bioprinting assembly 102 through the hole 140. The radiation source 116 and the aiming aid 118 are aligned with an opening 142 in the printhead housing 106 so that their operation during use of the bioprinting system 100 is unobstructed by the printhead housing 106.

Referring to FIG. 3, the bioprinting assembly 102 has an electronics assembly 144 electrically connected to each dispensing outlet 138. The electronics assembly 144 is configured to move each dispensing outlet 138 between its respective open and closed configurations.

The electronics assembly 144 has an electrical port 146 configured to electrically connect the electronics assembly 144 to a control system 150 (discussed below). The electronics assembly 144 also has an electrical connector 148 that is capable of being electrically connected to other electrical equipment that is internal or external to the bioprinting assembly 102. It is envisaged that the electronics assembly 144 may or may not include the electrical connector 148.

Referring to FIGS. 3 and 5, the cap 126 of each reservoir 120 is coupled in fluid communication to a pressure regulating system 152. The pressure regulating system 152 is configured to regulate/control the pressure within each of the reservoirs 120 and be coupled in fluid communication with an air compressor 154. It is also envisaged that, instead of the air compressor 154, the pressure regulating system 152 may be coupled in fluid communication with any other suitable source of pressurized gas known in the art. The pressure regulating system 152 may be an electro-pneumatic pressure regulating system. It is further envisaged that the pressure regulating system 152 may utilise any other suitable method of pressure regulation known in the art.

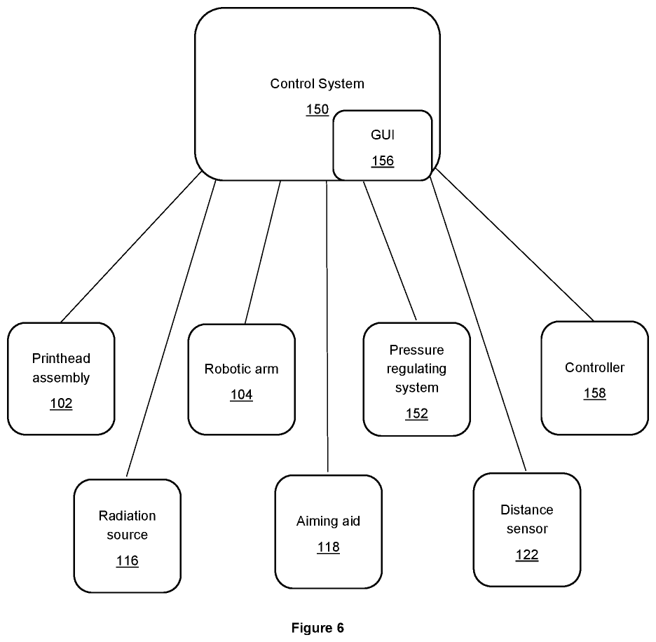

Referring to FIGS. 1 and 6, the bioprinting assembly 102, the robotic arm 104, the radiation source 116, the aiming aid 118, the distance sensor 122, and the pressure regulating system 152 are electrically connected to, and controlled by, a control system 150. The control system 150 has a graphical user interface (GUI) 156 that allows a user to input instructions into the control system 150. The GUI 156 will also display information to the user. The control system 150 is configured to allow a user to select and/or design a gel (discussed below) that is to be formed by the bioprinting system 100 via the GUI 156.

The control system 150 includes a non-transitory computer readable medium on which programs and algorithms for operating the bioprinting assembly 102, the robotic arm 104, and the pressure regulating system 152 are stored. It is envisaged that the non-transitory computer readable medium is located separately from the bioprinting system 100 and is electrically connected to the bioprinting system 100. It is also envisaged that the non-transitory computer readable medium may be provided with the bioprinting system 100.

A controller 158 (not shown in FIG. 1) is electrically connected to the control system 150. The controller 158 is configured to control movement of the robotic arm 104 to move and position the bioprinting assembly 102. FIG. 1 shows the GUI 156 as having a connector 160 in the form of a cable to which the controller may be connected. The user may therefore provide inputs into the control system 150 using one or both of the GUI 156 and controller 158. The controller 158 may be any suitable controller known in the art such as, for example, a gaming controller, joystick, a computer mouse, or a customized controller.

Liquids that are to be held in the reservoirs 120 may have to be kept within a certain temperature range and, therefore, the bioprinting assembly 102 may comprise a heater and/or a cooler to regulate the temperature within the housing 106.

Use and operation of the bioprinting system 100 will now be described.

### Priming the Reservoirs 120

Before a printing regime can be printed by the bioprinting system 100, one or more of the reservoirs 120 must be primed with the necessary liquids required for the print regime. When the bioprinting system 100 is turned on, the control system 150 is configured to increase the pressure within each reservoir 120 to a predetermined level using the pressure regulating system 152. To prime a reservoir 120 with a liquid, a user selects the reservoir 120 they wish to prime using the GUI 156 and the control system 150 subsequently controls the pressure regulating system 152 to reduce the pressure in the selected reservoir 120 to 0 kPa.

Once the selected reservoir 120 has been depressurized, a syringe, or the like, is removably coupled to the connector 134 of the priming fluid line 132 coupled to the depressurized reservoir 120. The liquid in the syringe can then be injected into the depressurized reservoir 120 through the respective priming fluid line 132 and reservoir inlet 130. After the depressurized reservoir 120 has been primed with liquid from the syringe, the syringe is decoupled from the connector 134 of the respective priming fluid line 132. Subsequently, the user uses the GUI 156 to confirm that the depressurized reservoir 120 has been primed, which causes the control system 150 to control the pressure regulating system 152 to increase the pressure in the depressurized reservoir 120 back to the predetermined pressure. As the pressure in the depressurized reservoir 120 increases, the liquid in the depressurized reservoir 120 flows into, and through, the respective dispensing fluid line 136 until it is stopped by the normally closed dispensing outlet 138 of the dispensing fluid line 136. At this stage, the reservoir 120 has been primed. To prime further reservoirs 120, the above methods steps are repeated.

The reservoirs 120 are primed with the necessary liquids required to complete a particular print regime. For example, the reservoirs 120 may be primed with bio-inks, radiation curable/photosensitive bio-inks, activators, cell-inks, cell-culture solutions, or utility solutions, all of which are described below.

### Designing a Printing Regime and Commencing Printing

After the necessary reservoirs 120 have been primed, the user designs/selects a printing regime to be printed to the site of a subject by the bioprinting system 100.

In an embodiment, the printing regime may form a gel to the site of the subject (i.e., patient) by dispensing bio-ink from the bioprinting assembly 102 that subsequently crosslinks to form a hydrogel. Bio-ink dispensed from the bioprinting assembly 102 can be crosslinked by dispensing an activator from the bioprinting assembly 102 onto the dispensed bio-ink. Alternatively, if the bio-ink is photosensitive or radiation curable, the dispensed bio-ink can be crosslinked by illuminating the dispensed bio-ink with radiation, such as, for example, UV radiation.

The GUI 156 allows a user to select and/or design a printing regime to be printed by the bioprinting system 100. The GUI 156 allows a user to select/design a printing regime based on the dimensions of a patient's wound. There are several ways in which the printing regime may be selected/designed.

### First Exemplary Method of Designing and Printing a Gel

According to one embodiment, a gel to be formed by the bioprinting system 100 can be designed by inputting the required dimensions of the gel into the control system 100 via the GUI 156.

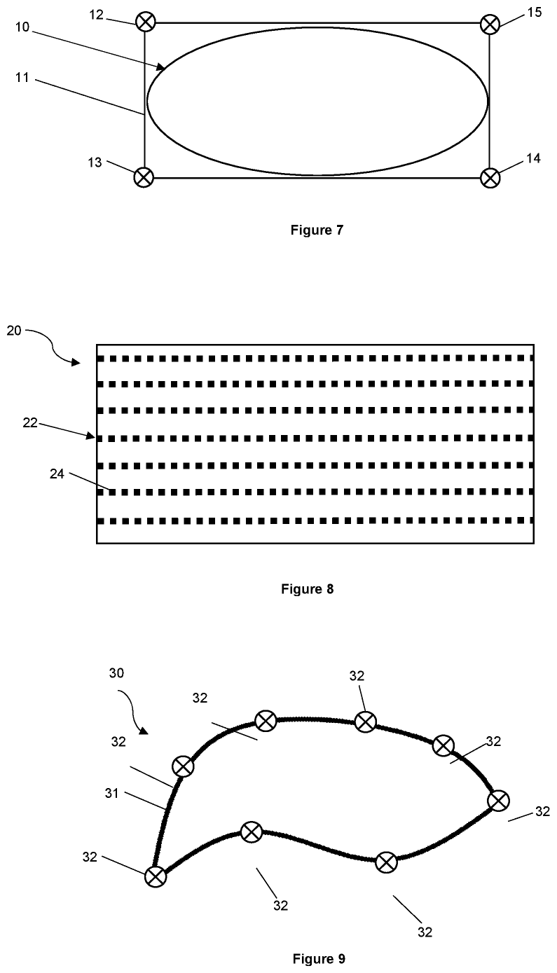

FIG. 7 shows a wound 10 in the skin of a patient and a box 11 that approximates the shape of, and encompasses, the wound 10. The box 11 may be visualized by the user. FIG. 8 shows a gel 20 having a substantially rectangular shape that is to be formed over the wound 10 by the bioprinting system 100.

Knowing the dimensions of the wound 10, the user uses the GUI 156 to input dimensions for the gel 20 that are larger than that of the wound 10 so that, when the gel 20 is formed using the bioprinting system 100, the formed gel 20 entirely covers the wound 10. After the dimensions for the gel 20 have been input into the control system 150, the control system 150 is configured to divide the gel 20 into a number of rows 22 each having one or more fly-by-points 24 (see FIG. 8). The fly-by-points 24 are specific points at which the control system 150 is triggered to control the dispensing system 114 to dispense fluid over the wound 10. The spacing between adjacent rows 22 and adjacent fly-by-points 24 in each row 22 determines the resolution of the gel 20 to be formed by the bioprinting system 100. The user may select the resolution for the gel 20 through the GUI 156 when designing the gel 20. The smaller the spacings, the higher the resolution of the gel 20 formed by the bioprinting system 100. The spacing between adjacent rows 22 and adjacent fly-by-points 24 in each row 22 may or may not be uniform. After the dimensions and the resolution for the gel 20 have been input into the control system 150, the user confirms the design of the gel 20 via the GUI 156.

Before the gel 20 can be formed by the bioprinting system 100, the bioprinting assembly 102 is moved to a starting position, which is a position that the bioprinting assembly 102 must initially be at so that the gel 20 is correctly aligned with and covers the wound 10 when formed. In this embodiment, the starting position is the top left corner 12 of the box 11, however, it is also envisaged that the other corners 12-15 of the box 11 may be used as the starting position.

To move the bioprinting assembly 102 to the starting position, the user uses the GUI 156 to turn on the aiming aid 118 and set the robotic arm 104 into a “free mode”. The free mode allows the user to manually move the bioprinting assembly 102 using the handles 108. The user then manually moves the bioprinting assembly 102 so that the laser of the aiming aid 118 is roughly pointing at the top left corner 12 of the box 11.

When positioning the bioprinting assembly 102 at the starting position, the user also manually positions the bioprinting assembly 102 so that the base 103 of the bioprinting assembly 102 is a predetermined distance from the printing surface. This predetermined distance may be measured using the distance sensor 122. In this case, the control system 150 may use the distance sensor 122 to measure the distance between the base 103 of the bioprinting assembly 102 and the printing surface and display the distance on the GUI 156. The control system 150 may also provide haptic feedback to the user, an auditory alarm, and/or a visual indication on the GUI 156 once the base 103 of the bioprinting assembly 102 is at the predetermined height from the printing surface. Alternatively, the predetermined height may be determined by a visual inspection performed by the user.

After the bioprinting assembly 102 has been manually positioned roughly at the starting position, the user uses the GUI 156 to take the robotic arm out of ‘free mode’. Subsequently, the user uses the controller 158 to more accurately position the bioprinting assembly 102 at the starting position. Once the bioprinting assembly 102 has been more accurately positioned at the starting position, the user turns off the aiming aid 118 through the GUI 156. At this stage, the user commences the printing regime via the GUI 156.

### Second Exemplary Method of Designing and Printing a Gel

The substantially rectangular gel 20 to be formed by the bioprinting system 100 may also be designed by mapping at least three corners of the wound 10.

According to this embodiment, the user moves the bioprinting assembly 102 manually and/or with the controller 158 (as described above) so that the aiming aid 118 is pointing at the top left corner 12 of the box 11 (see FIG. 7) and the base 103 of the bioprinting assembly 102 is at the predetermined distance from the printing surface (as described above). The user then uses the GUI 156 to map the top left corner 12 of the box 11. Mapping the top left corner 12 of the box 11 involves the control system 150 recording the spatial position of the bioprinting assembly 102 and the robotic arm 104. After the top left corner 12 has been mapped, the user repeats the above steps to map the bottom left corner 13 and the bottom right corner 14 of the box 11.

After the user has mapped the corners 12-14 of the box, the control system 150 is configured to design a rectangular gel 20 having dimensions larger than the wound 10 so that, when the gel 20 is formed by the bioprinting system 100, the formed gel 20 entirely covers the wound 10. The control system 150 also divides the designed gel 20 into a number of rows 22, each having one or more fly-by-points 24 as described above. The user can adjust the resolution of the designed gel 20 (i.e., the spacing between the adjacent rows 22 and adjacent fly-by-points 24 in each row 22) using the GUI 156 if needed. The user can then turn off the aiming aid 118 and commence the printing regime via GUI 156.

In this embodiment, the user does not have to move the bioprinting assembly 102 to a starting position before commencing the printing regime. This is because the control system 150 uses the mapped corners 12-14 as a spatial reference when forming the gel 20.

Although the method of designing the gel 20 has been described by mapping the corner 12-14 of the box 11, it will also be appreciated that the gel 20 can be designed by mapping any three corners 12-15 of the wound 10.

### Third Exemplary Method of Designing and Printing a Gel

The bioprinting system 100 can also be used to print irregularly shaped gel.

FIG. 9 shows a wound 30 in the skin of a patient having a periphery 31. Located on the periphery 31 is a plurality of aiming points 32. To design a gel to cover the wound 30, the user moves the bioprinting assembly 102 manually and/or with the controller 158 (as described above) so that the aiming aid 118 is pointing at one of the aiming points 32 and the base 103 of the bioprinting assembly 102 is at the predetermined distance from the printing surface (as described above). The user then uses the GUI 156 to map the aiming point 32, as described above. After the aiming point 32 has been mapped, the user repeats the above steps to map the remaining aiming points 32.

After the user has mapped all of the aiming points 32, the control system 150 is configured to design a gel having an irregular shape and size so that, when the gel is formed by the bioprinting system 100, the gel entirely covers the wound 30. The control system 150 also divides the designed gel into a number of rows 22, each having one or more fly-by-points 24 as described above. The user can adjust the resolution of the designed gel (i.e., the spacing between the adjacent rows 22 and adjacent fly-by-points 24 in each row 22) using the GUI 156 if needed. The user can then turn off the aiming aid 118 and commence the printing regime via GUI 156.

In this embodiment, the user does not have to move the bioprinting assembly 102 to a starting position before commencing the printing regime. This is because the control system 150 uses the mapped aiming points 32 as a spatial reference when forming the gel.

The aiming points 32 are arbitrarily chosen by the user. The user may decide to use more or less aiming points 32 when designing a gel using this method. It will be appreciated that increasing the number of aiming points 32 on the periphery 31 of the wound 30 will result in the control system 150 designing a gel having a shape that more accurately matches the shape of the wound 30.

### Fourth Exemplary Method of Designing and Printing a Gel

According to another method, a scaled image of a wound may be displayed on the GUI 156, which would allow the user to trace the periphery of the wound on the GUI 156. The control system 150 is configured to use the trace of the periphery of the wound to design a gel having a shape and size so that, when the gel is formed by the bioprinting system 100, the gel entirely covers the wound. The control system 150 divides the designed gel into a number of rows 22, each having one or more fly-by-points 24 as described above. The user can adjust the resolution of the designed gel (i.e., the spacing between the adjacent rows 22 and adjacent fly-by-points 24 in each row 22) using the GUI 156 if needed.

In this embodiment, after the gel has been designed, the control system 150 displays a starting position for the bioprinting assembly 102 on the GUI 156. The user then moves the bioprinting assembly 102 manually and/or with the controller 158 (as described above) so that the aiming aid 118 is distance at the starting position and the base 103 of the bioprinting assembly 102 is at the predetermined height from the printing surface. The user can then turn off the aiming aid 118 and commence the printing regime via GUI 156.

For each of the methods described above, it will be appreciated that the aiming aid 118 is offset from each of the dispensing outlets 138. The control system 150 accounts for the offset between the aiming aid 118 and each dispensing outlet 138 so that the printed gel 20 is correctly aligned with and covers the wound 10.

Although it has been described above that the gel is designed after the reservoirs 120 are primed, it is also envisaged that the gel may be designed before the reservoirs 120 are primed. If the reservoirs 120 are primed after the gel has been designed, the control system 150 may be configured to determine which reservoirs 120 need to be primed with a particular fluid and with what volume so that the bioprinting system 100 can complete forming the designed gel without the need for re-priming any of the reservoirs 120 during printing.

### Forming the Designed Gel

Once the user has commenced the printing regime, the control system 150 controls the bioprinting system 100 to dispense the required fluids at each of the fly-by-points 24 to form the designed gel. The designed gel is printed layer by layer and each layer is printed row 22 by row 22 by dispensing the required fluid at each fly-by-point 24 in each row 22. The number of layers forming the gel may be selected by the user when designing the gel and may be dependent on the depth of the wound of the patient. The rows 22 and fly-by-points 24 of the designed gel are spaced so that gel formed at each fly-by-point 24 in a layer merges with gel formed at adjacent fly-by-points 24 in the same layer so that the layer is at least substantially continuous and does not have any gaps or holes. The gel forming each layer merges with the gel of adjacent layers.

To dispense a particular liquid from the bioprinting assembly 102 at a fly-by-point 24, the control system 150 positions the bioprinting assembly 102 using the robotic arm 104 so that the dispensing outlet 138 of the reservoir 120 holding the particular liquid is aligned with the specific fly-by-point 24. The control system 150 then moves the respective dispensing outlet 138 to the open configuration and the pressure within the reservoir 120 forces the liquid within the reservoir 120 to be dispensed/ejected from the dispensing outlet 138. Once the required volume of the particular liquid has been dispensed from the respective dispensing outlet 138, the control system 150 moves the dispensing outlet 138 back to the closed configuration to prevent further liquid being dispensed from the dispensing outlet 138.

It will be appreciated that dispensing liquid from a reservoir 120 will reduce the pressure in the reservoir 120. Accordingly, after liquid has been dispensed from a reservoir 120 and the respective dispensing outlet 138 is moved to the closed configuration, the control system 150 controls the pressure regulating system 152 to re-pressurize the reservoir 120 to a predetermined pressure.

As the control system 150 maintains the base 103 of the bioprinting assembly 102, and therefore the dispensing outlets 138, a predetermined distance from the printing surface, the bioprinting system 100 provides a non-contact method of printing a liquid to a printing site.

The volume of liquid dispensed from the reservoirs 120 may be preset in the control system 150. However, the control system 150 may be configured to control the volume of the liquid dispensed from a particular reservoir 120 depending on the liquid contained in the reservoir 120 and the gel to be formed. Alternatively, the user may control the volume of the liquid dispensed from the bioprinting assembly 102 either through the control system 150 or manually through the GUI 156 when designing a gel.

The bioprinting system 100 may be configured to dispense/eject nanolitres of liquid from each reservoir 120. However, increasing and decreasing the pressure within a reservoir 120 will increase and decrease the flow rate of liquid through the corresponding dispensing outlet 138, respectively. Increasing and decreasing the period of time that the dispensing outlet 138 is in the open configuration will increase and decrease the volume of liquid dispensed from the dispensing outlet 138, respectively. Accordingly, it will be appreciated that the volume of liquid dispensed from a dispensing outlet 138 can be varied by varying the pressure within the respective reservoir 120 and varying the period of time that the dispensing outlet 138 is in the open configuration.

As the control system 150 moves the bioprinting assembly 102 over the surface of the patient's skin (i.e. the printing surface) using the robotic arm 104, the control system 150 uses the distance sensor 122 to control the robotic arm 104 to maintain the base 103 of the bioprinting assembly 102 at the predetermined distance from the surface of the patient's skin while forming the gel. The control system 150 is therefore able to maintain the base 103 of the bioprinting assembly 102 at a predetermined height from an uneven printing surface (e.g., the surface of the patient's skin) while moving the bioprinting assembly 102 over the uneven printing surface using the robotic arm 104, which avoids the bioprinting assembly 102 contacting the printing surface. This allows the bioprinting system 100 to more accurately and repeatably dispense fluid at each of the fly-by-points 24 when forming the gel.

The control system 150 can dispense liquid at each fly-by-point 24 in a row 22 while continuously moving the bioprinting assembly 102. The control system 150, therefore, does not need to stop the bioprinting assembly 102 at each of the fly-by-points 24 in a row 22 to dispense liquid. Accordingly, being able to continuously move the bioprinting assembly 102 and dispense liquid may provide a relatively fast method for forming a gel.

The control system 150 is able to position that bioprinting assembly 102 in any orientation using the robotic arm 104. The control system 150 is therefore able to position the bioprinting assembly 102 such that the dispensing outlets 138 are facing upwards. The bioprinting system 100 may be able to print with the dispensing outlets 138 facing upwards. This may be possible due to the pressure within the reservoirs 120. When a dispensing outlet 138 is facing upwards and is moved to its open configuration, the pressure within the respective reservoir 120 may be sufficient enough to eject fluid within the reservoir 120 out through the open dispensing outlet 138 onto a printing surface positioned above the dispensing outlet 138. In such a case, the pressure within the reservoirs 120 will have to be sufficient enough to eject fluid from the respective dispensing outlet 138 with enough force to reach the printing surface, which is position above the dispensing outlet 138 at a predetermined distance. For at least similar reasons, the bioprinting system 100 may be able to print with the dispensing outlets 138 facing sideways. Accordingly, it will be appreciated that the bioprinting assembly 102 may be able to print in any orientation, which may make printing onto hard to reach areas simpler.

In order to print with the dispensing outlets 138 facing upwards, it will be appreciated that liquid within the reservoirs 120 must be prevented from flowing away from the dispensing outlets 138. In an embodiment, the internal diameter of the reservoirs 120 may be small enough so that the pressure within the reservoirs 120, together with the small diameter of the reservoirs 120, prevents fluid flowing away from the dispensing outlets 138, when the dispensing outlets 138 are facing upwards.

### Drop-on-Drop Method

According to one embodiment, the bioprinting system 100 may print a gel using a drop-on-drop method. In this method, at least one reservoir 120 is primed with a bio-ink and at least one reservoir 120 is primed with an activator. The control system 150 controls the robotic arm 104 to move the bioprinting assembly 102 to each of the fly-by-points 24. The control system 150 is configured to dispense a drop of bio-ink at each fly-by-point 24 in a row 22 and then dispense a drop of activator at each of the fly-by-points 24 in the same row 22 to form hydrogel before moving onto the next row 22.

To form a gel using the drop-on-drop method described above, it will be appreciated that there must be a minimum of two reservoir 120 and that the radiation source 116 will not be needed. It is envisaged that multiple reservoirs 120 may be primed with bio-ink and that multiple reservoirs 120 may be primed with an activator. In this case, for example, when all the bio-ink has been dispensed from one reservoir 120, the control 150 would then dispense bio-ink from another reservoir 120. This will reduce the need to pause the printing regime to re-prime a reservoir 120.

It is also envisaged that the reservoirs 120 may be primed with different types of liquids. If the reservoirs 120 are primed with different liquids, the bioprinting system 100 may be able to form a gel having layers of different materials, layers that include different cells and/or medicaments, and/or different liquids printed/deposited between each layer of the gel.

### Radiation Curing Method

According to another embodiment, the bioprinting system 100 may form a gel using a UV/radiation curing method. In this method, at least one reservoir 120 is primed with a radiation curable bio-ink (e.g., rhCollagen). The control system 150 controls the robotic arm 104 to move the bioprinting assembly 102 to each of the fly-by-points 24. The control system 150 is configured to dispense a drop of radiation curable bio-ink at each of the fly-by-points 24 in a row 22 and then illuminate the dispensed bio-ink with the radiation source 118 to form hydrogel before moving onto the next row 22. To illuminate dispensed radiation curable bio-ink with the radiation source 116, the control system 150 moves the bioprinting assembly 102 using the robotic arm 104 so that the radiation source 116 is aligned with the dispensed bio-ink. After the gel has been formed, the control system 150 may be configured to illuminate the gel with the radiation source 116 by controlling the robotic arm 104 to move the bioprinting assembly 102 and, therefore, the radiation source 116 over the gel. This is to further cure the formed gel.

To form a gel using the radiation curing method described above, it will be appreciated that a minimum of one reservoir 120 is required. It is envisaged that multiple reservoirs 120 may be primed with bio-ink. In this case, when all the bio-ink has been dispensed from one reservoir 120, the control 150 would then dispense bio-ink from another reservoir 120. This will reduce the need to pause the printing regime to re-prime a reservoir 120.

It is also envisaged that the reservoirs 120 may be primed with different types of liquids. If the reservoirs 120 are primed with different liquids, the bioprinting system 100 may be able to form a gel having layers of different materials, layers that include different cells and/or medicaments, and/or different liquids printed/deposited between each layer of the gel.

### First Practical Example of Using the Bioprinting System 100

According to one practical example of using the bioprinting system 100, one or more of the reservoirs 120 may be primed with a bio-ink that includes cells and/or one or more of the reservoirs 120 may be primed with a suspension that includes cells. Accordingly, the gel that is subsequently formed into/over a patient's wound using this bio-ink and/or suspension will have cells that can be adsorbed by the patient and aid and accelerate healing of the wound.

In this example, the gel may be formed using the drop-on-drop or radiation curing methods described above and the cells used may be autologous cells and/or any other suitable cells known in the art.

### Second Practical Example of Using the Bioprinting System 100

According to another practical example of using the bioprinting system 100, the reservoirs 120 may be primed with different liquids depending on the depth of the patients wound. The patient's wound may be deep enough to expose different tissue types. In this case, the reservoirs 120 may be primed with different liquids so that the gel formed by the bioprinting system 100 has different gel layers formed at different depths within the wound. Having different gel layers formed at different depths within the wound of a patient may aid and accelerate healing of the patient's wound.

In this example, the gel may be formed using the drop-on-drop or radiation curing methods described above and the cells used may be autologous cells and/or any other suitable cells known in the art.

As an example, the wound of a patient may extend through the epidermis and the dermis of the patient. Accordingly, with the bioprinting system 100 it may be possible to form gel layers proximate the dermis containing dermis cells and then form gel layers proximate the epidermis containing epidermis cells. The dermis and epidermis cells may be autologous cells.

Accordingly, the bioprinting system 100 may deposit healthy cells into a wound of a patient by forming a three-dimensional (3D) gel containing cells and/or medicaments in the wound, which may assist with healing the wound. Further, part of this 3D gel may become part of the patient's skin at the wound site.

Although the bioprinting system 100 has been described above for designing and forming a gel over a wound of a subject (i.e., patient), it is also envisaged that the bioprinting system 100 may be used to print a liquid to a site of the subject using the same method described above. Such liquids may include cell and/or medicaments. Sites that the bioprinting system 100 may be used to print to include acute wounds (e.g., burns), chronic wounds (e.g., diabetic ulcers), cartilage, and muscles.

Although the radiation source 116 has been described as an array of UV LEDs, it is envisaged that other sources of radiation may be used as the radiation source 116. If this is the case, the bio-ink must be chosen/designed so that it will crosslink when exposed to the particular source of radiation chosen for the radiation source 116.

Although priming of the reservoirs 120 has been described and illustrated with reference to removably coupling a syringe to the connectors 134 of the priming fluid lines 132, it will be appreciated that the reservoirs 120 may be primed using other methods. For example, the reservoirs 120 may be primed by:
coupling a container containing a liquid to the fluid line 132 of a reservoir 120 and using a pump to pump liquid from the container into the reservoir 120;
coupling a syringe to the connector 134 of the fluid line 132 of a reservoir 120 as described above and automatically actuating the syringe to inject the content of the syringe into the reservoir 120;
sampling liquids from multiple liquid containers using a sample loading system, such as the sample loading system described and illustrated in the Applicant's International Patent Application No PCT/AU2019/051336, the contents of which are incorporated herein by reference in its entirety; or
coupling a syringe to a reservoir 120 using a means that creates a sterile fluidic connection, for example by using a septum, and using a desired method to draw fluid from the syringe into the reservoir 120.

It is also envisaged that the set of reservoirs 112 may be a cartridge that is removable from the bioprinting assembly 102. In this case, an empty cartridge may be removed from the bioprinting assembly 102 and replaced with a new cartridge. The reservoirs 120 forming the removable cartridge would be removably coupled to the pressure regulating system 152 and respective dispensing fluid lines 136. The reservoirs 120 forming the removable cartridge may be primed with the necessary liquids before being removably coupled to the pressure regulating system 152 and respective dispensing fluid lines 136.

Gels may be formed using methods other than the drop-on-drop and radiation curing methods described above. For example, a gel may be formed by:
ionic transfer from the wound of the patient to the bio-ink to form the gel;
thermal gelation, where body heat from the patient, or heat from an external heat source, is used to crosslink the bio-ink to form the hydrogel; and
using other radiation sources/wavelengths to crosslink the bio-ink to form the hydrogel.

Example bio-inks that may be used with the bioprinting system 100 are described in the Applicant's International Patent Application No PCT/AU2019/050767, the contents of which are incorporated herein by reference in its entirety.

It is also envisaged that the gel may be designed by detecting the wound of the patient. Examples of how the wound could be detected are provided below.
The distance sensor 122 could be used to generate a “map” of the patient. “Patient mapping” could include mapping the outline and/or depth of the patient's wound. This could also include mapping the surface of the patient's body around the wound. The map of the patient could be generated before or during the printing regime and be used to design the gel to be formed by the bioprinting system 100. The distance sensor 122 used to generate the map could be an ultrasonic sensor, optical sensor, camera, eddy current sensor, or combinations thereof.
A map of the patient's wound may be generated by disposing a material or object around the wound that can be detected by a suitable sensor to generate an outline of the wound. For example, the material or object disposed around the wound may omit a signal that may be detected by a suitable sensor. These signals may include, but are not limited to, visible light, infrared light, UV light, X-ray radiation, gamma radiation, magnetic radiation, or the like.

### Bio-Ink

In the present specification, bio-ink is defined as an aqueous solution of one or more types of macromolecule in which cells may be suspended or housed. Upon activation or crosslinking, it creates a hydrogel structure having its physical and chemical properties defined by chemical and physical composition of the bio-ink. Macromolecules are defined as an array of both synthetic and natural polymers, proteins and peptides. Macromolecules may be in their native state or chemically modified with amine or thiol-reactive functionalities.

Synthetic macromolecules may include:
Polysaccharides, such as polymers containing fructose, sucrose or glucose functionalities;
Non-ionic polymers, such as poly(ethylene glycol) (PEG), poly(hydroxyethyl methacrylate (PHEMA), poly(ε-caprolactone) (PCL), poly(vinyl alcohol) (PVA), poly(vinylpyrrolidone) (PVP), poly(NIPAAM) and poly(propylene fumarate) (PPF) and derivatives;
Polyelectrolytes—polymers that carry either positive or negative charge, amphoteric as well as zwitterionic polymer;
Polypeptides—a single linear chain of many amino acids (a minimum of 2 amino acids), held together by amide bonds; and
Nucleobase containing synthetic polymers—polymers with nucleobase (adenine, thymine, guanine or cytosine) repeating units.

Natural macromolecules may include:
Polysaccharides, such as alginate, chitosan, gellan gum, hyaluronic acid, agarose and glycosaminoglycan;
Proteins, such as gelatin, fibrin and collagen;
DNA and Oligonucleotides, such as single stranded DNA (ssDNA), double stranded DNA (dsDNA) DNAzymes and Aptamers; and
Basement membrane extracts.
Amine-reactive functionalities may include: aldehyde, epoxy, N-hydroxysuccinimide (NHS) and 2-vinyl-4,4-dimethylazlactone (VDM).
Thiol-reactive functionalities may include: alkenes, alkynes, azides, halogens and cyanates.
The bio-ink used and found suitable was alginate (at 2 w/v %) dissolved in calcium free DMEM supplemented with 10 v/v % FCS, L-glutamine and sodium pyruvate.
Bio-ink with dispersed SK-N-BE(2) neuroblastoma cells is referred to as bio-ink containing cells.

### Activator

In the present specification, an activator is an aqueous solution comprising of either small molecules or macromolecules which interact with the bio-ink to form a hydrogel structure. The composition of the activator can be altered to control the physical properties of the resulting hydrogel. The type of activator used is highly dependent on the macromolecules used as well as the intended crosslinking process.

Activators can be selected from:
Inorganic salts such as calcium carbonate, calcium chloride, sodium chloride, magnesium sulphate. sodium hydroxide and barium chloride;
Photoinitiators such as 2,2-dimethoxy-2-phenylacetophenone (DMPA) and Irgacure;
Polyelectrolytes—polymers that carry an opposite charge to the macromolecules in the bio-ink. It could be cationic, anionic, amphoteric and zwitterionic;
Polypeptides—a single linear chain of many amino acids (a minimum of 2 amino acids), held together by amide bonds;
DNA linker—macromolecules carrying nucleotides or DNA sequences which complement those present on the bio-ink's macromolecules; and
Natural or synthetic macromolecules carrying amine or thiol groups, either natively or through chemical modifications.
The activator used for the alginate bio-ink was calcium chloride at 4 w/v % dissolved in MilliQ water.

### Crosslinking or Gelation

This is the process whereby individual macromolecular chains are linked together by the activator to form a hydrogel. The crosslinking process can be classified to either chemical or physical crosslinking. Physical crosslinking or non-covalent crosslinking may include:
Ionic crosslinking—crosslinking via the interaction of the opposite charges present in the macromolecule and the activator. The activator may include charged oligomers, ionic salt and ionic molecule;
Hydrogen bonds—crosslinking via the electrostatic attractions of polar molecules. In this case, the macromolecule and the activator are carrying polar functionalities;
Temperature crosslinking—crosslinking via the rearrangement of the macromolecular chains as a response to change in temperature (heating or cooling); and
Hydrophobic interaction or van der Waals force.

Chemical or covalent crosslinking involves chemical reactions between the macromolecule and the activator. The type of reactions may include:
Photocrosslinking whereby the crosslinking reaction is promoted by UV or light irradiation;
Michael-type addition reaction between thiols and vinyl-carrying macromolecules in aqueous media;
Schiff base reaction between amino and aldehyde groups;
Diels-alder reaction;
Click chemistry;
Aminolysis reaction to active ester group; and
Enzyme crosslinking.

Examples of other bio-ink and activator combinations are set out in the Table below:

| Bio-Ink | Activator |
|---|---|
| Positively charged polyelectrolyte | Negatively charged polyelectrolyte |
| (e.g. poly (trimethylammonium) | (e.g. poly(sulfonate), |
| or poly(guanidium) | poly(carboxylic acid) |
| Fluorenylmethoxycarbonyl | Phosphate buffer solution |
| (Fmoc) polypeptide | Cell culture medium |
| Thiol-reactive macromolecules | Photoinitiator and/or thiol-containing |
| (e.g. PEG-diacrylate, hyaluronic | macromolecules (e.g. bis-thiol-PEG) |
| acid maleimide) | Thiol-containing polypeptides (e.g. |
| | bis-cysteine functionalised peptide) |
| Amine-reactive macromolecules | Amine-containing polypeptides (e.g. |
| (e.g. PEG-co-Poly(benzaldehyde), | bis-amine functionalised peptide, |
| aldehyde-alginate | gelatin, collagen) |
| Charged polysaccharides(e.g. | Inorganic salts (e.g. calcium |
| alginate and gellan gum) | chloride, barium chloride). |
| Macromolecules containing | Macromolecules containing the |
| nucleobase (e.g. Adenine) | corresponding nucleobase pair |
| | (e.g. Thymine) |

### Cell-Ink

In the present specification, cell-inks are an aqueous solution of one or more type of molecules or macromolecules in which cells are to be and remain evenly suspended throughout the 3D bio-printing process. The concentration of the cell-ink is optimised to prevent cells from settling but still maintains high cell viability.

Cell-link can be selected from:
Small molecules such as glycerol
Macromolecules such as Ficoll™, dextran, alginate, gellan gum, methylcellulose; and poly(vinylpyrrolidone) (PVP).
Ficoll™ is a neutral, highly branched, high-mass, hydrophilic polysaccharide which dissolves readily in aqueous solutions. Ficoll™ radii range from 2-7 nm and is prepared by reaction of the polysaccharide with epichlorohydrin. Ficoll™ is a registered trademark owned by GE Healthcare companies.
The cell-ink used was Ficoll™ 400 (at 10 w/v %) dissolved in PBS.
Cell-ink with dispersed SK-N-BE(2) neuroblastoma cells is referred to as cell-ink containing cells.
Gellan gum is a water-soluble anionic polysaccharide produced by the bacterium Sphingomonas elodea (formerly Pseudomonas elodea).

### Cell-Culture Solutions

In the present specification, cell-culture solutions are liquids that come into contact with the cultured cells and are suitable for various cell-related works. The preparation process includes careful analysis of the salt and pH balance, incorporation of only biocompatible molecules and sterilisation.

Some of the cell culture solutions include:
Cell culture medium such as Dulbecco's Modified Eagle Medium (DMEM), Minimum Essential Media (MEM), Iscove's Modified Dulbecco's Medium (IMDM), Media 199, Ham's F10, Ham's F12, McCoy's 5A and Roswell Park Memorial Institute (RPMI) medium;
Growth supplements such as foetal calf serum (FCS), epidermal growth factor (EGF), basic fibroblast growth factor (bFBF), fibroblast growth factor (FBF), endothelial cell growth factor (ECGF), insulin-like growth factor 1 (IGF-1) and platelet-derived growth factor (PDGF);
Biological buffers such as PBS, HEPES and CHES;
Chelating and stabilizing solutions; and
Sterilized MilliQ water.

### Cell-Culture Conditions

Cells and the 3D tissue culture models can be incubated, cultured and maintained using standard cell culture techniques. The 3D tissue culture models comprising the cells encapsulated in the hydrogel mold can be incubated under conditions to allow or maintain cell growth or spheroid formation. Incubation is typically carried out at about 37° C. with a CO2 level of 5% for at least 24 hours for most animal and human cell lines. It will be appreciated that incubation can be carried out at any suitable conditions, temperature and time duration that allows growth, maintenance or spheroid formation of the type of cell or cells in the hydrogel mold.

### Utility Solutions

Utility solutions are defined as the solutions which do not come into contact with the cells but are used to clean and sterilize the reservoirs 120, priming fluid lines 132, dispensing fluid lines 136, dispensing outlets 138 and all surfaces of the bioprinting system 100 exposed to the cells. In other words, the utility solutions are cleaning fluids. These solutions may include:
Ethanol at the correct concentration;
Sterile MilliQ water;
Cell culture medium;
Detergent; and
Hydrogen peroxide solution (2 w/v % maximum concentration).

### Preparation of Bio-Ink

Initially, bio-ink is prepared by mixing the right type and amount of macromolecules in the appropriate cell-culture solution. After achieving homogeneity, the blank bio-ink is sterilised via both UV irradiation and filtration (0.22 μm filter). The bio-ink is then kept at 4° C. until further usage.

### Preparation of Cells

Harvest cells by washing with PBS. Aspirate PBS. Add trypsin and incubate at 37° C. to dissociate cells from flask surface. Add tissue culture media to collect dissociated cells into a tube. Centrifuge cells, aspirate supernatant and resuspend pellet in fresh media. Perform cell count by mixing equal volumes of cell suspension and trypan blue stain. Perform calculation to determine the cell concentration. Desired numbers of cells then can be added to bio-ink, cell-ink or added to cell culture solutions.

### Preparation of Activators

The correct type and amount of molecules were dissolved in the appropriate cell-culture solution. The resulting solution was sterilised via UV irradiation and filtration prior to use.

### Preparation of Cell-Ink

The correct type and amount of molecules were dissolved in the appropriate cell-culture solution. After achieving homogeneity, the resulting solution was sterilised via UV irradiation and filtration prior to use. The cell-ink was then kept at room temperature until further use.

### Cell Harvesting

Cultured cells of interest at certain confluency are harvested by following the already established protocols. To make up the bio-ink or cell-ink containing cells, harvested cells are resuspended at the correct cell concentration to give 252 million cells/ml concentration in 200 μl of bio-ink or cell-ink. The resulting cell pellets are then redispersed in the correct volume of bio-ink or cell-ink. The bio-ink or cell-ink containing cells is then ready for use in the 3D bio-printer.

### Cell Types

3D tissue culture models such as spheroids can be prepared from any suitable cell type including adherent cells such as mammalian liver cells, gastrointestinal cells, pancreatic cells, kidney cells, lung cells, tracheal cells, vascular cells, skeletal muscle cells, cardiac cells, skin cells, smooth muscle cells, connective tissue cells, corneal cells, genitourinary cells, breast cells, reproductive cells, endothelial cells, epithelial cells, fibroblast, neural cells, Schwann cells, adipose cells, bone cells, bone marrow cells, cartilage cells, pericytes, mesothelial cells, cells derived from endocrine tissue, stromal cells, stem cells, progenitor cells, lymph cells, blood cells, endoderm-derived cells, ectoderm-derived cells, mesoderm-derived cells, or combinations thereof.

Additional cell types may include other eukaryotic cells (e.g. chinese hamster ovary), bacteria (e.g. Helicobacter pylori), fungi (e.g. Penicillium chrysogenum) and yeast (e.g. Saccharomyces cerevisiae).

The cell line SK-N-BE(2) (neuroblastoma cells) has been used successfully in the process to produce 3D tissue culture models under a range of conditions. It will be appreciated that other cell lines would be expected to perform as required in 3D tissue models produced by the process developed. Other cell lines used include DAOY (human medulloblastoma cancer cells), H460 (human non-small lung cancer) and p53R127H (human pancreatic cancer cells). Other cell lines that may be suitable are listed on 088 and 089.

The bioprinting system 100 allows gels to be printed over the wound having a uniform thickness and a more consistent deposition of cells and/or medicaments over the wound compared to other known methods. The bioprinting system 100 can also more accurately apply/print cells and with a higher resolution compared to other known methods. Gels having a consistent deposition of cells/or medicaments may improve healing of a wound of a patient. The bioprinting system 100 may also allow different biological matter and/or medicaments to be printed to a wound of a patient that may improve healing of the wound.

### Distance Sensor

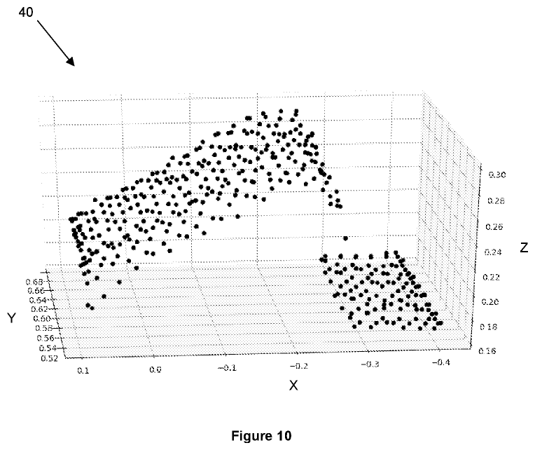

FIG. 10 shows a three-dimensional plot 40 of an uneven surface taken by a scan using the distance sensor 122 of the bioprinting system 100. Preferably, the distance sensor 122 is utilised to produce a similar three-dimensional plot of a wound of a subject. The 3D plot of a subject's wound or data thereof may be used by the computer or controller(s) of the bioprinting system 100 to determine the location on the subject to which bioprinted fluids are to be applied and/or the amounts of bioprinting fluid that are to be dispensed at each point of the location. The 3D plot may also permit a doctor or expert to review the surface of the subject being scanned in detail prior to any action being taken.

### Experimental Study

An experiment using a bioprinting system according to the present disclosure was carried out on wounds of a number of pigs. Multiple hydrogel formulations containing cells were printed in 20×20×0.46 mm patches into full thickness wounds of the same size in a pig model.

The experimental study used the following method to treat a wound on a pig using bio-printing of autologous cells. In the first step, the clinician created a full thickness excisional wound of 20×20 mm on the pig. In the second step, the piece of skin removed from the pig to create an excisional wound was disaggregated using an enzyme solution to produce a 250 μL cell suspension of mixed population autologous cells, including keratinocytes, fibroblasts, and melanocytes. Thirdly, the autologous mixed cell population suspension was mixed with 250 μL of activator to produce a 500 μL activator cell suspension. The 500 μL activator cell suspension was transferred to a surgical syringe using a pipette. The surgical syringe containing the 500 μL activator cell suspension was connected to the printhead reservoir luer lock to create a fluidic connection between the surgical syringe and the printhead reservoir. The 500 μL activator cell suspension was loaded into the reservoir by pushing the syringe plunger into the barrel. The surgical syringe was disconnected from the luer lock completing the loading of the 500 μL activator cell suspension into the printhead reservoir.

A 0.5 mL volume of bio-ink was loaded into a different reservoir before the activator cell suspension using the same method as above for treatment of each wound.

After the bio-ink and activator cell suspension were loaded into the reservoirs, the system was pressurized to a pressure of 60 kPa using the pressure regulators. 200 droplets were dispensed as waste from the nozzles to remove any excess air bubbles in the printing system. The 6-axis robot was switched to “free” mode and the clinician manually positioned the printhead near a corner of the 20×20 mm wound. The robot arm positional controller was used to finely adjust the location of the printhead using the laser aiming aid just prior to printing. Once positioned, printing into the wound commenced by scanning the printhead and printing a single row of bio-ink droplets and subsequently a row of activator cell suspension droplets to form a cross-linked hydrogel. A plurality of these rows were printed to create a single layer of hydrogel containing cells in the base of the wound. This process was repeated to form a second layer on top of the first layer. After printing was completed, the wound was dressed with a wound dressing.

The printing process and in situ gelation dynamics, studied in twenty printed wounds across four pigs, were found to enable sufficient structural integrity and wound integration to reliably control spatial positioning of cells within the wound site. Assessment of cells post-print showed negligible impact of the printing and gelation processes on cell viability. Preliminary data investigating wound outcomes post intervention showed promising indications of the viability of 3D printing to deliver cells and matrix effectively to a wound environment.

It has been determined that biological 3D printing has the potential to transform acute surgical intervention for skin wounds. Currently the technology is most suitable for use in a clinic environment.

Although the invention has been described with reference to a preferred embodiment, it will be appreciated by persons skilled in the art that the invention may be embodied in many other forms. It will be appreciated by persons skilled in the art that numerous variations and/or modifications may be made to the technology as shown in the specific embodiments without departing from the spirit or scope of technology as broadly described. The present embodiments are, therefore, to be considered in all respects as illustrative and not restrictive.

### INDEX OF FEATURES

10—Wound
11—Box
12—Top left corner of box 11
13—Bottom left corner of box 11
14—Bottom right corner of box 11
15—Top right corner of box 11
20—Gel
22—Rows
24—Fly-by-points
30—Wound
31—Periphery of wound 30
32—Aiming points
40—3D plot
100—Bioprinting system
102—Bioprinting assembly of bioprinting system 100
103—Base of bioprinting assembly 102
104—Robotic arm of bioprinting system 100
106—Housing of bioprinting assembly 102
108—Handles of bioprinting assembly 102
110—Access panel of printhead housing 106
112—Set of reservoirs of bioprinting assembly 102
114—Dispensing system of bioprinting assembly 102
116—Radiation source of bioprinting assembly 102
118—Aiming aid of bioprinting assembly 102
120—Reservoirs of set of reservoirs 112
122—Distance sensor of bioprinting assembly 102
124—Longitudinal axis of reservoirs 120
126—Cap of reservoirs 120
128—Reservoir outlet of reservoirs 120
130—Reservoir inlet of reservoirs 120
132—Priming fluid line of reservoirs 120
134—Connector of priming fluid lines 132
136—Dispensing fluid line of dispensing system 114
137—Housing of dispensing outlets 138
138—Dispensing outlet of dispensing fluid line 136
140—Hole of printhead housing 106
142—Opening of printhead housing 106
144—Electronics assembly of printhead housing 106
146—Electrical port of electronics assembly 144
148—Electrical connector of electronics assembly 144
150—Control system of bioprinting system 100
152—Pressure regulating system of bioprinting system 100
154—Air compressor
156—Graphical user interface (GUI)
158—Controller
160—Connector
162—Trolley
170—Mounting base of robotic arm 104
171—First axis of rotation of robotic arm 104
172—Second axis of rotation of robotic arm 104
173—Third axis of rotation of robotic arm 104
174—Fourth axis of rotation of robotic arm 104
175—Fifth axis of rotation of robotic arm 104
176—Sixth axis of rotation of robotic arm 104
178—Mounting connector of robotic arm 104 to bioprinting assembly 102

1. A bioprinting system for printing a liquid to a site of a subject, the bioprinting system comprising:
a bioprinting assembly configured to dispense the liquid to the site of the subject, the bioprinting assembly having at least one reservoir configured to hold the liquid to be dispensed by the bioprinting assembly, and a loading mechanism in fluid communication with the reservoir configured to load the reservoir with the liquid prior to printing onto the site of the subject, wherein the loading mechanism provides a sterile fluidic connection and comprises a one way inlet that permits the liquid to be loaded into the reservoir and prevents fluid from exiting the reservoir via the one way inlet.

2. The bioprinting system of claim 1, wherein the subject is a patient, the site is a wound in the subject's skin, and the liquid dispensed by the bioprinting assembly forms a gel over the wound.

3. The bioprinting system of claim 1, further comprising:
a robotic arm coupled to the bioprinting assembly, the robotic arm configured to move and position the bioprinting assembly over the site; and
a control system configured to control the bioprinting assembly and the robotic arm

4. The bioprinting system of claim 3, wherein the bioprinting assembly further comprises a distance sensor configured to monitor the distance between the bioprinting assembly and the site of the subject, and wherein the control system is configured to use distance information from the distance sensor to control the robotic arm to maintain the bioprinting assembly at a predetermined distance from the site while printing the liquid.

5. The bioprinting system of claim 1, wherein the loading mechanism comprises any one or more of a check valve, a septum, and a luer lock.

6. The bioprinting system of claim 1, wherein the loading mechanism has a priming fluid line providing a fluid communication between the one way inlet and the reservoir.

7. The bioprinting system of claim 1, wherein the one way inlet is configured to be removably coupled to a syringe, preferably wherein the one way inlet has a connector configured to be removably coupled to the syringe.

8. The bioprinting system of claim 1, where the bioprinting assembly comprises a plurality of reservoirs and a plurality of loading mechanisms each in fluid communication with a respective reservoir.

9. The bioprinting system of claim 1, wherein the at least one reservoir has a dispensing fluid line that is configured to dispense fluid from the at least one reservoir.

10. The bioprinting system of claim 9, wherein the dispensing fluid line has a dispensing outlet having:
an open configuration that allows liquid to be dispensed from the at least one reservoir; and
a closed configuration that prevents liquid from being dispensed from the at least one reservoir.

11. The bioprinting system of claim 1, further comprising a pressure regulating system coupled in fluid communication with the at least one reservoir, the pressure regulating system configured to regulate pressure within the at least one reservoir.

12. The bioprinting system of claim 1, wherein the bioprinting assembly further comprises an aiming aid that is configured to assist with positioning the bioprinting assembly.

13. The bioprinting system of claim 1, wherein the liquid to be dispensed from the bioprinting assembly includes reagents and activators and is preferably selected from bio-inks, radiation curable bio-inks, activators, cell-inks, and cell-culture solutions.

14. The bioprinting system of claim 1, wherein the bioprinting assembly further comprises a radiation source configured to cure a radiation curable fluid dispensed by the bioprinting assembly.

15. The bioprinting system of claim 1, wherein the robotic arm and the bioprinting assembly are configured such that the bioprinting assembly can be manoeuvred to print the liquid onto the site of the subject in any desired orientation.

16. A method of forming a gel over a site of a subject using the bioprinting assembly of claim 1, the method comprising:
a) dispensing a liquid from the bioprinting assembly to a point of the site;
b) dispensing an activator from the bioprinting assembly onto the dispensed liquid to form a hydrogel; and
c) repeating steps a) and b) at a plurality of different points of the site to form the gel over the wound.

17. The method of claim 16, wherein the liquid is a reagent selected from bio-inks, radiation curable bio-inks, activators, cell-inks, and cell-culture solutions.

18.-20 (canceled)

21. The method of claim 16, wherein the liquid includes cells and/or medicaments.

22. (canceled)

23. The method of claim 16, wherein a droplet size of the liquid is selected such that the liquid forms a gel at the site of the subject without movement due to gravity.

24. (canceled)

25. A method of priming a reservoir with a liquid, comprising:
a) providing the bioprinting system of claim 1;
b) connecting a container comprising the liquid to the loading mechanism in a sterile fluidic connection;
c) transferring the liquid from the container to the bioprinting assembly; and
d) priming the reservoir with the liquid.

26. The method of claim 21, wherein the liquid is a bio-ink or cell-ink and comprises cells, preferably,—the cells are autologous cells of the subject.

27. The method of claim 21, wherein the container is a syringe, and wherein transferring the liquid comprises injecting the liquid into the bioprinting assembly.

28. The method of claim 21, wherein the bioprinting system is provided in an operating theatre, and wherein the steps b) to d) are each performed within the operating theatre before the liquid is to be printed onto the site of the subject.

29. The method of claim 16, wherein the subject is a patient and the site is a wound in the subject's skin.
